(19) United States
(12) Patent Application Publication
(10) Pub. No.: US 2025/0011378 A1
(43) Pub. Date: Jan. 9, 2025

(54) NOVEL PROTEIN DERIVED FROM MUSSEL PERIOSTRACUM AND USE THEREOF

(71) Applicant: POSTECH RESEARCH AND BUSINESS DEVELOPMENT FOUNDATION, Pohang-si (KR)

(72) Inventors: Dong Soo HWANG, Pohang-si (KR); Hyung Bin KIM, Pohang-si (KR)

(73) Assignee: POSTECH RESEARCH AND BUSINESS DEVELOPMENT FOUNDATION, Pohang-si (KR)

(21) Appl. No.: 18/754,355

(22) Filed: Jun. 26, 2024

(30) Foreign Application Priority Data
Jul. 3, 2023 (KR) 10-2023-0085929

(51) Int. Cl. C07K 14/435; C09D 189/00

(52) U.S. Cl. CPC C07K 14/43504; C09D 189/00

(57) ABSTRACT

The present disclosure relates to a novel protein derived from mussel periostracum and a use thereof, and the mussel periostracum protein was isolated and purified, named Antifoulin, the entire sequence was derived, a recombinant antifouling protein was prepared therefrom, and an antifouling function was confirmed, and thus the protein can be usefully used as a coating model system for surface coating agents, especially biocompatible medical devices.

### CROSS-REFERENCE TO RELATED APPLICATIONS

This application is based on and claims priority from Korean Patent Application No. 10-2023-0085929, filed on Jul. 3, 2023, with the Korean Intellectual Property Office, the disclosure of which is incorporated herein in its entirety by reference.

### INCORPORATION BY REFERENCE OF SEQUENCE LISTING

The content of the electronically submitted sequence listing, file name: Q299497_sequence listing as filed.XML; size: 11,586 bytes; and date of creation: Jun. 24, 2024, filed herewith, is incorporated herein by reference in its entirety.

### TECHNICAL FIELD

The present disclosure relates to a novel protein derived from mussel periostracum and a use thereof.

### BACKGROUND

Fixative marine animals such as Balanidae, algae, and marine microorganisms adhere to surfaces of marine structures, ships, etc. that are exposed to the ocean for a long period of time. Such fouling causes surface corrosion, which causes problems such as weakened durability or energy consumption in the case of ships. In addition, medical devices such as stents that are inserted into the human body are prone to durability problems due to adhesion of microorganisms or biomolecules in the body. Therefore, the development of surface coating technology is continuously progressing to prevent such fouling.

In the medical field, coating agents for medical devices are currently made of synthetic polymer-based materials such as polytetrafluoroethylene (PTFE). However, due to the development of technology, there are more cases where devices remain in the human body for a long period of time, and thus the coating surface requires biocompatibility and requires performance such as corrosion of the device surface or maintenance of durability through an antifouling effect.

Biopolymers such as proteins are promising as biocompatible antifouling materials. Since proteins with low-friction properties, such as lubricin have glycosylation as the main principle, it is nearly impossible to mass-produce these proteins for industrial use with sugar chain control using current technology. In addition, there are not many known examples of biopolymers with an antifouling function. Therefore, it will be important for future use in medical coatings to discover and suggest new precursor materials capable of implementing antifouling surfaces using biopolymers rather than synthetic chemicals.

Living organisms build hard exoskeletons, such as insect cuticles and mollusk shells, to protect soft tissues. For example, the hard shell of mussel serves to protect the soft inner body from predators and dynamic mechanical shocks between high and low tides. The shell basically consists of the periostracum, the prismatic layer, and the nacreous layer, and the prismatic layer and the nacreous layer exhibit strong and tough characteristics due to a brick-and-mortar microstructure formed by calcium carbonate mineralization. In contrast, the periostracum, which is an inconspicuous outermost layer, has been found to have properties such as abrasion resistance, antifouling and compartmentalization despite low stiffness (Biofouling, 2006; 22 (4): 251-259).

Accordingly, the present inventors purified a novel protein called antifoulin from a mussel periostracum protein to obtain sequences and completed the present disclosure by synthesizing a recombinant antifoulin protein.

### SUMMARY

The present disclosure has been made in an effort to provide a novel protein derived from mussel periostracum, a polynucleotide encoding the protein, an expression vector including the polynucleotide, and a host cell including the expression vector.

The present disclosure has also been made in an effort to provide a method for preparing the protein.

The present disclosure has also been made in an effort to provide a coating liquid composition including the protein and a coating method using the same.

An embodiment of the present disclosure provides a protein derived from mussel periostracum including an amino acid sequence as set forth in SEQ ID NO: 1 or an amino acid sequence functionally equivalent thereto, a polynucleotide encoding the protein, an expression vector including the polynucleotide, and a host cell including the expression vector.

Another embodiment of the present disclosure provides a method for preparing a protein derived from mussel periostracum including culturing a host cell under conditions suitable for protein expression; and recovering the protein.

Yet another embodiment of the present disclosure provides a coating liquid composition with excellent abrasion resistance and antifouling properties including a protein derived from mussel periostracum and a method for coating using the same.

Another embodiment of the present disclosure provides a method of providing an antifouling coating, the method comprising applying a coating composition comprising the protein derived from mussel periostracum to a surface of an article.

According to the embodiments of the present disclosure, the present disclosure relates to a novel protein derived from mussel periostracum and a use thereof, and the mussel periostracum protein was isolated and purified, named Antifoulin, the entire sequence was derived, a recombinant antifouling protein was prepared therefrom, and abrasion resistance and an antifouling function were confirmed, and thus the protein can be usefully used as a coating model system for surface coating agents, especially biocompatible medical devices.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is an image of glass, polytetrafluoroethylene (PTFE), or periostracum observed through an atomic force microscope.

FIG. 2 is a plot between a lateral force and an applied force of glass, polytetrafluoroethylene (PTFE) or periostracum exposed to air, in which the slope of the graph represents a friction coefficient (u).

FIG. 3 is a plot between a lateral force and an applied force of hydrated glass, polytetrafluoroethylene (PTFE) or periostracum, in which the slope of the graph represents a friction coefficient (u).

FIG. 4 is a graph showing a friction coefficient for a silica tip of glass, polytetrafluoroethylene (PTFE), or periostracum.

FIG. 5 is a graph analyzing roughness of glass, polytetrafluoroethylene (PTFE), or periostracum.

FIG. 6 is an SDS-PAGE gel image confirming the expression of recombinant Antifoulin-1 according to an IPTG concentration in E. coli (BL21) transformed with an Antifoulin-1 cDNA sequence.

FIG. 7 is an SDS-PAGE gel image confirming the expression of recombinant Antifoulin-1 in transformed E. coli cells for each culture time after treatment with 0.5 mM IPTG.

FIG. 8 is an SDS-PAGE gel image showing a result of separation and purification using solvents in E. coli expressing recombinant Antifoulin-1.

FIG. 9 is a diagram showing an antifouling effect on E. coli and a water contact angle of a surface coated with recombinant Antifoulin-1.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, the present disclosure will be described in detail.

The present disclosure provides a novel protein derived from mussel periostracum.

The present inventors purified a constituent precursor protein from mussel periostracum, named antifoulin, derived the entire sequence of the protein, and confirmed that the protein had lubricating and antifouling functions.

In a specific embodiment, the present disclosure provides a protein including an amino acid sequence as set forth in SEQ ID NO: 1 or an amino acid sequence functionally equivalent thereto. In an embodiment, the protein may be a protein including amino acid sequences as set forth in SEQ ID NOs: 2 to 5. In addition, the protein may include all peptides with various amino acid sequences added to the N-terminus or C-terminus of the amino acid sequence. In addition, the protein may further include an amino acid sequence designed for a specific purpose to increase a targeting sequence, a tag, labeled residues, a half-life or peptide stability. In addition, as long as the activity of the protein is not changed, variant proteins in which some amino acids in the amino acid sequences as set forth in SEQ ID NOs: 1 to 5 are mutated by addition, substitution, deletion, etc. may also be included in the scope of the protein provided by the present disclosure. Amino acid exchanges at the protein and peptide level that do not entirely alter the activity of the protein are known in the art. In some cases, the amino acids may be modified by phosphorylation, sulfation, acrylation, glycosylation, methylation, farnesylation, and the like.

In addition, the present disclosure provides a polynucleotide including a base sequence encoding the protein.

In a specific embodiment, a gene encoding the protein having the amino acid sequence as set forth in SEQ ID NO: 1 may be a polynucleotide having a base sequence as set forth in SEQ ID NO: 6. In addition, a gene encoding the protein having the amino acid sequence as set forth in SEQ ID NO: 2 may be a polynucleotide having a base sequence as set forth in SEQ ID NO: 7. A gene encoding the protein having the amino acid sequence as set forth in of SEQ ID NO: 3 may be a polynucleotide having a base sequence as set forth in SEQ ID NO: 8. A gene encoding the protein having the amino acid sequence as set forth in SEQ ID NO: 4 may be a polynucleotide having a base sequence as set forth in SEQ ID NO: 9. A gene encoding the protein having the amino acid sequence as set forth in SEQ ID NO: 5 may be a polynucleotide having a base sequence as set forth in SEQ ID NO: 10.

In the present disclosure, the base sequence constituting the polynucleotide may be in the form in which base sequences capable of encoding the amino acid sequences as set forth in SEQ ID NOs: 1 to 5 or base sequences encoding various amino acid sequences that may be added to the N-termini or C-termini of the amino acid sequences as set forth in SEQ ID NOs: 1 to 5 may be added to the 5′-termini or 3′-termini of the base sequence encoding the amino acid sequence as set forth in SEQ ID NO: 1. Preferably, the base sequence constituting the polynucleotide may be in the form in which base sequences as set forth in SEQ ID NOs: 6 to 10 capable of encoding the amino acid sequences as set forth in SEQ ID NOs: 1 to 5 or base sequences encoding various amino acid sequences that may be added to the N-termini or C-termini of the amino acid sequences as set forth in SEQ ID NOs: 1 to 5 may be added to the 5′-termini or 3′-termini of the base sequences as set forth in SEQ ID NOs: 6 to 10. In addition, a polynucleotide including a base sequence having homology with the base sequence may also be included in the scope of the polynucleotide provided by the present disclosure, preferably a polynucleotide including a base sequence having 80% or more homology, more preferably a polynucleotide including a base sequence having 90% or more homology, and most preferably a polynucleotide including a base sequence having 95% or more homology.

Meanwhile, various modifications may be made in a coding region within the range that does not change an amino acid sequence of the protein or an active fragment thereof expressed from the coding region. In addition, various mutations may occur even in parts other than the coding region within the range that does not affect gene expression. For example, as long as the polynucleotide encodes a protein with activity equivalent to that of the protein, one or more bases may be mutated by substitution, deletion, insertion, or a combination thereof. When preparing a nucleotide sequence by chemical synthesis, the synthesis method may use synthesis methods well-known in the art, for example, methods described in the literature (Engels and Uhlmann, Angew Chem IntEd Engl, 37:73-127, 1988), and may include triester, phosphite, phosphoramidite and H-phosphate methods, PCR and other autoprimer methods, oligonucleotide synthesis methods on solid supports, etc.

Further, the present disclosure provides an expression vector including the polynucleotide.

As used herein, the term “expression vector” refers to a gene construct that includes a gene insert so that a target protein may be expressed in an appropriate host cell and includes a required regulatory element which is operably linked so that the gene insert is expressed. The expression vector includes expression regulatory elements such as a start codon, a stop codon, a promoter, an operator, etc., and the start codon and the stop codon are generally considered to be part of the nucleotide sequence encoding the polypeptide, need to act in a subject when the gene construct has been administered, and need to be in frame with the coding sequence. The promoter of the vector may be constitutive or inductive.

As used herein, the term “operably linked” means that a nucleic acid sequence encoding a target protein or RNA is functionally linked to a nucleic acid expression regulatory sequence to perform a general function. For example, a nucleic acid sequence encoding a protein or RNA may be operably linked to a promoter to affect the expression of a coding sequence. The operable linkage to the expression vector may be prepared using a gene recombination technique well-known in the art, and site-specific DNA cleavage and linkage may be used with enzymes and the like which are generally known in the art.

In addition, the expression vector may include a signal sequence to promote the separation of the protein from a cell culture medium. A specific initiation signal may also be required for efficient translation of an inserted nucleic acid sequence. These signals include an ATG start codon and adjacent sequences. In some cases, it is required to provide an exogenous translation regulatory signal, which may include the ATG start codon. These exogenous translation regulatory signals and start codons may be a variety of natural and synthetic sources. Expression efficiency may be increased by introduction of appropriate transcription or translation enhancers.

In the present disclosure, the expression vector is not particularly limited as long as the expression vector may produce the protein provided by the present disclosure by expressing the polynucleotide, but may be a vector capable of replicating and/or expressing the polynucleotide in eukaryotic or prokaryotic cells, including mammalian cells (e.g., human, monkey, rabbit, rat, hamster, mouse cells, etc.), plant cells, yeast cells, insect cells, or bacterial cells (e.g., E. coli, etc.), preferably a vector that is operably linked to an appropriate promoter so that the polynucleotide may be expressed in a host cell and includes at least one selection marker. Since the expression level, modification, and the like of the protein vary depending on a host cell, it is preferable to select and use the host cell most suitable for the purpose as the expression vector. In a specific embodiment, the expression vector may be pET-28 (+).

Further, the present disclosure provides a host cell including the expression vector.

The host cell is not particularly limited as long as the host cell may produce the peptide by expressing the polynucleotide, but may be bacterial cells such as E. coli, Streptomyces, and Salmonella Typhimurium; yeast cells such as Saccharomyces cerevisiae and Schizosaccharomyces pombe; fungal cells such as Pichia pastoris; insect cells such as Drosophila and Spodoptera Sf9 cells; animal cells such as CHO, COS, NSO, 293, Bow's melanoma cells; or plant cells. In a specific embodiment, the host cell may be E. coli.

In addition, a method of introducing and transforming the expression vector provided by the present disclosure into a host cell is not particularly limited as long as the expression vector may produce the protein of the present disclosure, but may use a CaCl₂) precipitation method, a Hanahan method for enhancing efficiency by using a reduced material, dimethyl sulfoxide (DMSO) in the CaCl₂) precipitation method, an electroporation method, a calcium phosphate precipitation method, a protoplast fusion method, a stirring method using silicon carbide fiber, an agrobacterium-mediated transformation method, a transformation method using PEG, dextran sulfate, lipofectamine, and drying/inhibition-mediated transformation methods, and the like.

Another embodiment of the present disclosure provides a method for preparing a protein derived from mussel periostracum including culturing a host cell under conditions suitable for protein expression; and recovering the protein.

As another method, a method for preparing a protein derived from mussel periostracum of the present disclosure includes (a) obtaining a polynucleotide encoding amino acid sequences as set forth in SEQ ID NOs: 1 to 5; (b) cloning the obtained polynucleotide to obtain an expression vector; (c) introducing the obtained expression vector into a host cell; and (d) culturing the host cell and recovering a protein derived from mussel periostracum including the amino acid sequences as set forth in SEQ ID NOs: 1 to 5 from the host cell.

As used here, the term “culturing” means a method for growing microorganisms under environmental conditions that are appropriately artificially controlled. In the present disclosure, the method of culturing the host cell may be performed using methods widely known in the art. Specifically, the culture is not particularly limited as long as the protein derived from mussel periostracum of the present disclosure may be expressed and produced.

The medium used for culture may meet the requirements of a specific strain in an appropriate manner while controlling a temperature, pH, etc. under aerobic conditions in a general medium containing appropriate carbon sources, nitrogen sources, amino acids, vitamins and the like. As the carbon source that may be used, mixed sugars of glucose and xylose are used as a main carbon source, and in addition, the carbon source includes sugars and carbohydrates such as sucrose, lactose, fructose, maltose, starch and cellulose, oils and fats such as soybean oil, sunflower oil, castor oil, and coconut oil, fatty acids such as palmitic acid, stearic acid, and linoleic acid, alcohols such as glycerol and ethanol, and organic acids such as acetic acid. These materials may be used individually or as a mixture. The nitrogen source that may be used may be used with inorganic nitrogen sources such as ammonia, ammonium sulfate, ammonium chloride, ammonium acetate, ammonium phosphate, ammonium carbonate, and ammonium nitrate; and organic nitrogen sources including amino acids and peptones such as glutamic acid, methionine, and glutamine, NZ-amine, meat extract, yeast extract, malt extract, corn steep liquor, casein hydrolyzates, fish or decomposition products thereof, and defatted soybean cake or decomposition products thereof. These nitrogen sources may be used alone or in combination. The medium may include monopotassium phosphate, dipotassium phosphate, and corresponding sodium-containing salts as a phosphate source. The phosphate source that may be used includes potassium dihydrogen phosphate or dipotassium hydrogen phosphate, or corresponding sodium-containing salts. In addition, as the inorganic compound, sodium chloride, calcium chloride, iron chloride, magnesium sulfate, iron sulfate, manganese sulfate, calcium carbonate, etc. may be used. Finally, in addition to the materials, required growth materials such as amino acids and vitamins may be used.

In addition, the step of recovering the protein derived from the mussel periostracum from the culture medium may be performed by methods known in the art. Specifically, the recovery method is not particularly limited as long as the protein derived from the mussel periostracum of the present disclosure may be recovered, but may use preferably centrifugation, filtration, extraction, spraying, drying, evaporation, precipitation, crystallization, electrophoresis, differential dissolution (e.g., ammonium sulfate precipitation), chromatography (e.g., ion exchange, affinity, hydrophobicity and size exclusion), etc.

Further, the present disclosure provides a coating liquid composition with excellent abrasion resistance and antifouling properties including a protein derived from mussel periostracum and a method for coating using the same.

In addition, the present disclosures provides a method of providing an antifouling coating, the method comprising applying a coating composition comprising the protein derived from mussel periostracum to a surface of an article.

According to the present disclosure, it was confirmed that a recombinant antifouling protein was produced by isolating the protein derived from mussel periostracum, and the protein was surface-coated to inhibit bacterial adhesion. Therefore, the protein of the present disclosure can be usefully used in the methods for preparing and coating the coating liquid composition with excellent abrasion resistance and antifouling properties.

Hereinafter, the present disclosure will be described in more detail through Examples and Experimental Examples.

These Examples and Experimental Examples are just illustrative of the present disclosure, and it will be apparent to those skilled in the art that it is not interpreted that the scope of the present disclosure is limited to these Examples and Experimental Examples.

### <Example 1> Purification and Sequence Analysis of Mussel Periostracum Protein

### <1-1> Separation of Mussel Periostracum

The inner periostracum layer of mussel (Mytilus californianus) was extracted using a surgical blade and stored at −80° C. until an experiment was performed.

### <1-1> Sequence Analysis of Mussel Periostracum Protein

A constituent precursor protein was purified from mussel periostracum and analyzed using liquid chromatography-mass spectrometry (LC-MS/MS).

Specifically, for tryptic proteolysis, periostracum was finely ground in liquid nitrogen and a sample was immersed in a 6 M urea and 0.4 M ammonium bicarbonate solution. Thereafter, tris(2-carboxyethyl)phosphine (TCEP) was added up to 5 mM in the solution and stirred for 60 minutes. The solution was added with Iodoacetamide 25 mM and cultured for 60 minutes in the dark. The solution was diluted 10-fold using a 40 mM ammonium bicarbonate solution and then degraded with trypsin at 37° C. for 16 hours to obtain a crude peptide.

To identify the constituent protein, LC-MS/MS analysis was performed using Easy n-LC (Thermo Fisher, San Jose, CA) and an LTQ Orbitrap XL mass spectrometer equipped with nanoelectrospray (Thermo Fisher, San Jose, CA). Extracted peptides obtained by trypsin degradation in the solution were separated on a C18 nanopore column (150 mm×0.1 mm, 3 μm pore size, Agilent). A mobile phase A for LC separation was 0.1% formic acid and 3% acetonitrile in deionized water, and a mobile phase B was 0.1% formic acid in acetonitrile. The chromatographic gradient was designed to increase linearly from 0% B to 40% B within 40 minutes, from 40% B to 60% B within 4 minutes, to 95% B within 4 minutes, and to 100% A within 6 minutes. The flow rate was maintained at 1500 nL/min. Mass spectra were acquired using a data-dependent acquisition (DDA) method with a full mass scan (350-1200 m/z) and 10 MS/MS scans. For MS1 full scan, OBILAB resolution was 15,000 and automatic gain control (AGC) was 2×10⁵. For MS/MS of LTQ, AGC was 1×10⁴. To identify the constituent proteins, the acquired spectra were compared with transcriptomes using PEAKS software.

As a result, the constituent proteins were named Antifoulin-1, Antifoulin-2, and Antifoulin-3, and the entire sequences of the proteins were derived as shown in Table 1. The gene sequence of each protein was shown in Table 2.

TABLE 1

| Name | Amino acid sequence | SEQ ID NO: |
|---|---|---|
| Antifoulin-1 | EKGQRGYSPRHNLKHYGSIEGRGLWLECGFSG | 1 |
| | DCNSYHRFNGGGGSGGRIGGVGYGYGIGSGYG | |
| | GYRNVGYGGYGIGSGYGYGIGSG | |
| Antifoulin-2 | WYGGWGSRGGRGGRGGGWGDGWSGGWGGS | 2 |
| (Variant 1) | RRYVGSGHVYVERQPWWGSYGGRGGGYGGG | |
| | YGGYSGGYGGYGGSSGGGGGYGGYGGYGGY | |
| | G | |
| Antifoulin-2 | WYGGWGSRGGRGGRGGGWGDGWSGGWGGS | 3 |
| (Variant 2) | RRYVGSGHVYVERQPWWGSYGRYGGRGGGY | |
| | GGGYGGYSGGYGGYGGYG | |
| Antifoulin-2 | WYGGWGSRGGRGGRGGGWGDGWSGGWGGS | 4 |
| (Variant 3) | RRYVGSGHVYVERQPWWGSYGRYGGRGDGY | |
| | GGYSGGYGGYGGYG | |
| Antifoulin-3 | DQGYGGGRSIGHVGGGRGLGSGGGAVRLVLG | 5 |
| | GGGGSGGGGEGKGDGRVWCTCRPGQCLKGEF | |
| | RLDKQCRLTKLFPWKWYTCCKVLKVTSRSYA | |
| | GGISGGARFGGGAGYGGGAGYFGGAGYGGRA | |
| | VYVVKSGYGGGSGYGGGAGYGKGSGHGGGA | |
| | GYGGGSGYGSGNSYIVVADGGSGGGSRGGY | |

TABLE 2

| Name | Gene sequence | SEQ ID NO: |
|---|---|---|
| Antifoulin-1 | gaaaaaggacaaagaggatatagtccaagacacaatctgaaacattatgg | 6 |
| | atcaatagaaggtagaggattgtggttagagtgtggattctctggcgactgt | |
| | aattcctatcacagattcaatggtggtggtggatctgggggcagaatcggc | |
| | ggtgtcggatatggttatggtattggttctggatacggagggtatagaaatg | |
| | ttggatatggtgggtatggcattggttccggatatgggtatggcattggttcc | |
| | ggata | |
| Antifoulin-2 | tggtatggaggatgggggagtcgcggtggacgcggtggacgcggtggt | 7 |
| (Variant 1) | ggatggggagatggatggagtggtggatggggaggtagtagaagatac | |
| | gtaggatccggacatgtatatgttgaaagacaaccttggtggggtagctat | |
| | ggtggtagaggcggtggatatggtggtggatatggtggttacagtggtgg | |
| | atatggtggatatggtggttccagtggtggaggcggtggatatggtggtta | |
| | tggtggatatggtggatatggt | |
| Antifoulin-2 | tggtatggaggatgggggagtcgcggtggacgcggtggacgcggtggt | 8 |
| (Variant 2) | ggatggggagatggatggagtggtggatggggaggtagtagaagatac | |
| | gtaggatccggacatgtatatgttgaaagacagccttggtggggtagctat | |
| | ggtagatatggtggtagaggcggtggatatggtggtggatatggtggttac | |
| | agtggtggatatggtggatatggtggatatggt | |
| Antifoulin-2 | tggtatggaggatgggggagtcgcggtggacgcggtggacgcggtggt | 9 |
| (Variant 3) | ggatggggagatggatggagtggtggatggggaggtagtagaagatac | |
| | gtaggatccggacatgtatatgttgaaagacagccttggtggggtagctat | |
| | ggtagatatggtggtagaggcgatggatatggtggttacagtggtggatat | |
| | ggtggatatggtggatatggt | |
| Antifoulin-3 | gaccaaggatatggtggtggaagaagcattgggcatgtcggtggtggca | 10 |
| | gaggattgggatcaggcggtggtgccgtaagattggtattaggcggagg | |
| | aggaggatccggcggtggtggagaaggaaaaggagatgggcgcgtgt | |
| | ggtgtacctgtcgtccaggacaatgtctaaaaggagaatttcgtttggaca | |
| | aacaatgcagacttaccaaattgtttccttggaaatggtatacatgctgcaa | |
| | ggtgttaaaagtaactagccgtagttacgctggaggaatcagtggtggag | |
| | ctagatttggaggtggggcaggatatgggggcggagcaggatatttcgg | |
| | cggagctggatatggtggcagagctgtatatgtagttaaatctggatatggt | |
| | ggcggatctggttatggaggaggagctggatatggtaaaggatctggtca | |
| | tggaggtggtgctggatatggaggaggatctggatatggcagtgggaatt | |
| | catatattgttgtggcagatggaggttccggtggaggttctcgtggaggat | |
| | attaatactaaatatgtttggtctgacaacacaaattatgtttaaaaatgaat | |

### <Example 2> Production of Recombinant Antifoulin Protein

### <2-1> Construct of Antifoulin Vector

Antifoulin-1 cDNA was inserted into NcoI and EcoRI restriction enzyme sites of a pET-28 (+) vector to prepare an antifoulin vector. The cDNA sequence was obtained through reverse transcription based on RNA extracted from periostracum cells. The pET-28 (+) vector includes trc, a promoter for E. coli expression to induce protein expression using IPTG.

### <2-2> Preparation of Transformant Producing Antifoulin

A transformant was prepared by transforming the antifoulin vector prepared in Example 2-1 into BL21 (DE3), a competent cell. An antifoulin vector solution was added to a competent cell solution and vortexed to induce transformation. Transformed colonies were selected using kanamycin.

### <2-3> Production and Isolation and Purification of Antifoulin

An antifoulin-transformed transformant E. coli was cultured in an LB medium (37° C.). When the absorbance of the E. coli culture medium at 600 nm reached 0.5, various concentrations of IPTG were added to induce protein expression. After addition of IPTG, the cells were cultured under the same temperature conditions for 5 to 16 hours. The cultured transformant was centrifuged at 4° C. and 10000 rpm, the culture medium was removed, and then the protein was purified using an elution buffer.

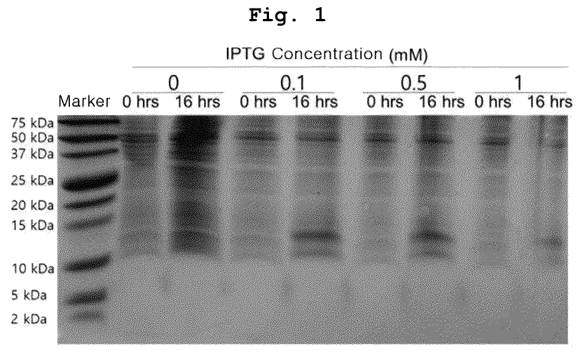

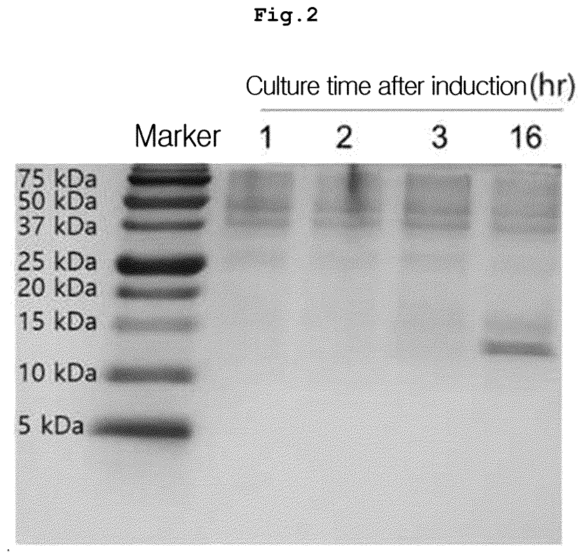

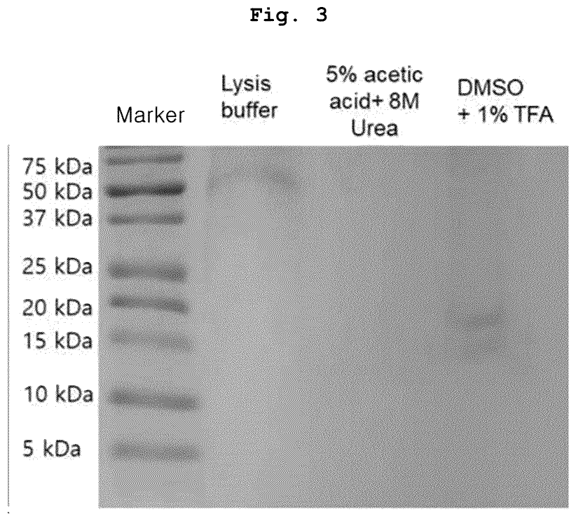

As a result, as shown in FIG. 1, it was confirmed that recombinant Antifoulin-1 was expressed at a concentration of 0.1 to 0.5 mM IPTG. In addition, as shown in FIG. 2, it was confirmed that when cultured for 16 hours after treatment with 0.5 mM IPTG, recombinant Antifoulin-1 was expressed. In addition, as shown in FIG. 3, it was confirmed that the Antifoulin-1 protein was purified when DMSO was used as an elution solvent.

### <Experimental Example 1> Confirmation of Lubricating Function of Mussel Periostracum

The roughness and friction characteristics of mussel periostracum were observed using an atomic force microscope.

Specifically, the friction coefficient was measured using an MFP-3D atomic force microscope (Asylum Research, Santa Barbara, CA) mounted on an inverted optical microscope. AppNano FORTA silicon tips (AC240TSA-R3) were used in the experiment, and a spring constant value was approximately 1.17 nN/nm, measured before each test. Samples were prepared on glass slides, and the samples submerged in water were cultured for 30 min immediately before starting measurement. The exposed samples were tested within 2 hours after removal of seawater. All images were recorded in an AC mode at a scan rate of 0.7 Hz.

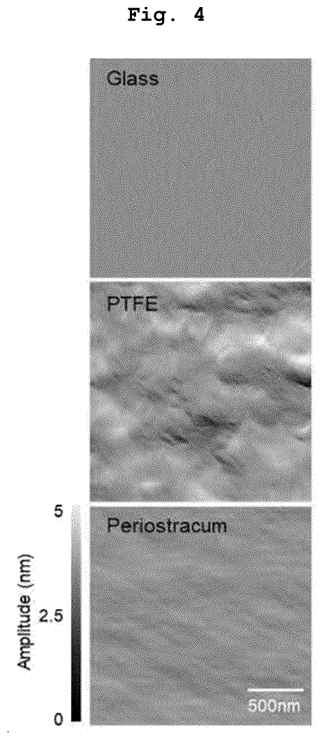

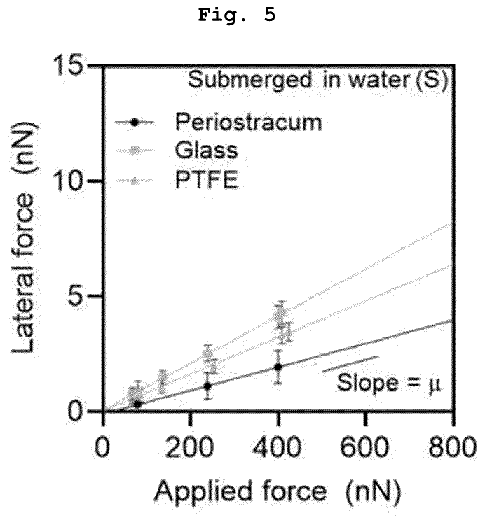

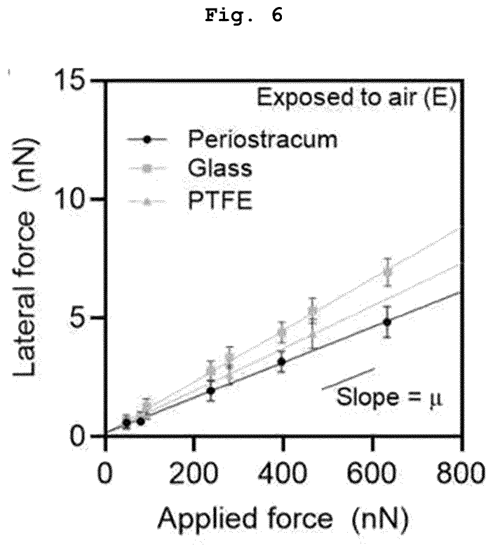

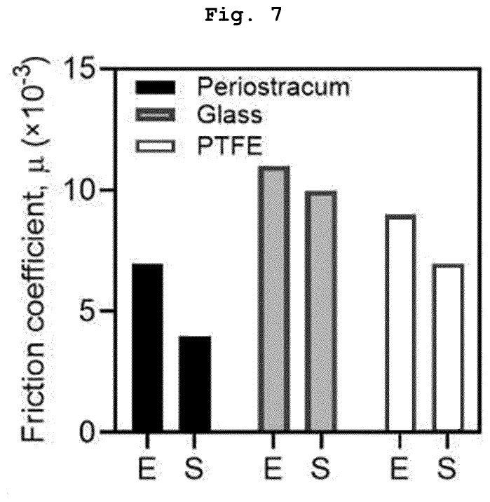

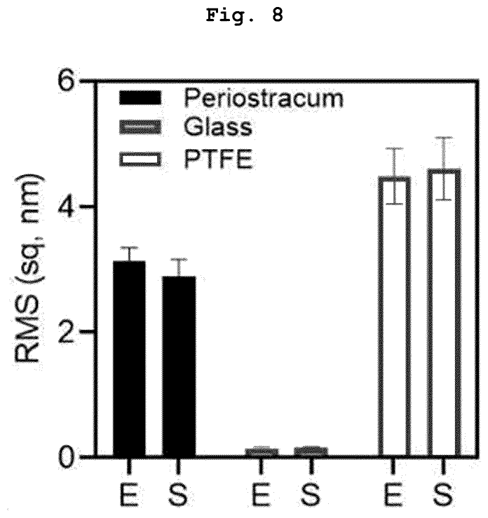

As a result, as shown in FIGS. 4 to 8, it was confirmed that the 0.66 and 0.77 times lower roughness and the friction coefficient (μ≈0.007) were exhibited when comparing the surfaces of hydrated glass and polytetrafluoroethylene (PTFE). The friction coefficient of periostracum was similar to the friction coefficient of articular cartilage. In addition, the friction coefficient in the hydrated state was 0.57 times lower than the friction coefficient in a state exposed to air. This suggests that lubrication occurs in an interfacial hydration layer.

### <Experimental Example 2> Confirmation of Surface Coating and Antifouling Function of Antifoulin

After performing surface coating using the Antifoulin-1 protein obtained in Example 2 above, the antifouling function was analyzed.

Specifically, the concentration of Antifoulin-1 protein was adjusted to 50 μg/ml through Bradford assay, and then the protein solution was placed on the surface and coated for 30 minutes. Thereafter, the surface was thoroughly washed with DMSO and water. E. coli was used to confirm the bacterial adhesion resistance on A ntifoulin-1-coated surface. E. coli was cultured in an LB medium at 37° C. and 200 rpm until the OD₆₀₀ value reached 0.1. A coated sample and an uncoated sample (control) were cultured in an E. coli solution for 16 hours at 37° C. and 200 rpm. After culturing, the samples were thoroughly rinsed with a PBS buffer (pH7) to remove the unadhering bacteria. The samples were transferred to tubes containing 1 ml of LB medium and then vortexed for 3 minutes to separate the adhering bacteria from the surface. The bacterial solution was serially diluted and cultured on an LB solid medium for 18 hours. Colonies were then counted to calculate bacterial CFU.

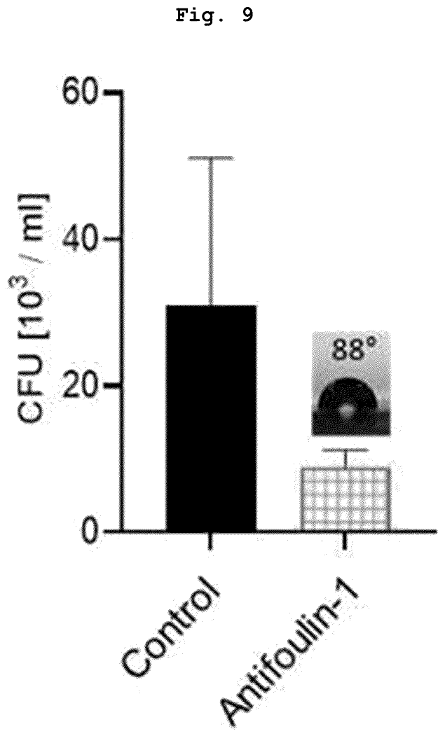

As a result, as shown in FIG. 9, it was confirmed that the number of colonies was significantly reduced in the sample coated with the antifoulin protein compared to the control.

The result indicates that the antifoulin protein has a property resistant to bacterial adhesion.

Meanwhile, when coated with antifoulin, a water contact angle was found to be 88°, which indicates that antifoulin is slightly hydrophobic, reflecting the water absorption of the periostracum.

The result suggests that the hydrophobic property of absorbing water may improve the water fluidity of the surface to prevent bacterial adhesion.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A protein comprising an amino acid sequence as set forth in SEQ ID NO: 1 or an amino acid sequence functionally equivalent thereto.

2. The protein of claim 1, wherein the protein includes amino acid sequences as set forth in SEQ ID NO: 2 to 5.

3. The protein of claim 1, wherein the protein is derived from mussel periostracum.

4. The protein of claim 1, wherein the protein has a resistance to bacterial adhesion.

5. The protein of claim 1, wherein the protein is encoded by the sequence as set forth in SEQ ID NO: 6.

6. The protein of claim 1, wherein the protein is encoded by the sequences as set forth in SEQ ID NOs: 7 to 10.

7. A method for preparing the protein of claim 1, comprising:
(1) culturing the host cell comprising an expression vector comprising a polynucleotide comprising a sequence encoding the protein under conditions suitable for protein expression; and
(2) recovering the protein.

8. A coating liquid composition comprising the protein of claim 1.

9. The coating liquid composition of claim 1, wherein the coating liquid composition has excellent abrasion resistance and antifouling properties.

10. A method of providing an antifouling coating, the method comprising: applying a coating composition comprising the protein of claim 1 to a surface of an article.


SEQUENCE LISTING (10 sequences)

SEQ ID NO: 1 - protein, 87 aa, Mytilus californianus
EKGQRGYSPRHNLKHYGSIEGRGLWLECGFSGDCNSYHRFNGGGGSGGRIGGVGYGYGIGSGYGGYRNVGYGGYGIGSGYGYGIGSG

SEQ ID NO: 2 - protein, 90 aa, Mytilus californianus
WYGGWGSRGGRGGRGGGWGDGWSGGWGGSRRYVGSGHVYVERQPWWGSYGGRGGGYGGGYGGYSGGYGGYGGSSGGGGGYGGYGGYGGYG

SEQ ID NO: 3 - protein, 77 aa, Mytilus californianus
WYGGWGSRGGRGGRGGGWGDGWSGGWGGSRRYVGSGHVYVERQPWWGSYGRYGGRGGGYGGGYGGYSGGYGGYGGYG

SEQ ID NO: 4 - protein, 73 aa, Mytilus californianus
WYGGWGSRGGRGGRGGGWGDGWSGGWGGSRRYVGSGHVYVERQPWWGSYGRYGGRGDGYGGYSGGYGGYGGYG

SEQ ID NO: 5 - protein, 184 aa, Mytilus californianus
DQGYGGGRSIGHVGGGRGLGSGGGAVRLVLGGGGGSGGGGEGKGDGRVWCTCRPGQCLKGEFRLDKQCRLTKLFPWKWYTCCKVLKVTSRSYAGGISGGARFGGGAGYGGGAGYFGGAGYGGRAVYVVKSGYGGGSGYGGGAGYGKGSGHGGGAGYGGGSGYGSGNSYIVVADGGSGGGSRGGY

SEQ ID NO: 6 - DNA, 263 bp, Mytilus californianus
  source 1..263: mol_type = genomic DNA
gaaaaaggacaaagaggatatagtccaagacacaatctgaaacattatggatcaatagaaggtagaggattgtggttagagtgtggattctctggcgactgtaattcctatcacagattcaatggtggtggtggatctgggggcagaatcggcggtgtcggatatggttatggtattggttctggatacggagggtatagaaatgttggatatggtgggtatggcattggttccggatatgggtatggcattggttccggata

SEQ ID NO: 7 - DNA, 270 bp, Mytilus californianus
  source 1..270: mol_type = genomic DNA
tggtatggaggatgggggagtcgcggtggacgcggtggacgcggtggtggatggggagatggatggagtggtggatggggaggtagtagaagatacgtaggatccggacatgtatatgttgaaagacaaccttggtggggtagctatggtggtagaggcggtggatatggtggtggatatggtggttacagtggtggatatggtggatatggtggttccagtggtggaggcggtggatatggtggttatggtggatatggtggatatggt

SEQ ID NO: 8 - DNA, 231 bp, Mytilus californianus
  source 1..231: mol_type = genomic DNA
tggtatggaggatgggggagtcgcggtggacgcggtggacgcggtggtggatggggagatggatggagtggtggatggggaggtagtagaagatacgtaggatccggacatgtatatgttgaaagacagccttggtggggtagctatggtagatatggtggtagaggcggtggatatggtggtggatatggtggttacagtggtggatatggtggatatggtggatatggt

SEQ ID NO: 9 - DNA, 219 bp, Mytilus californianus
  source 1..219: mol_type = genomic DNA
tggtatggaggatgggggagtcgcggtggacgcggtggacgcggtggtggatggggagatggatggagtggtggatggggaggtagtagaagatacgtaggatccggacatgtatatgttgaaagacagccttggtggggtagctatggtagatatggtggtagaggcgatggatatggtggttacagtggtggatatggtggatatggtggatatggt

SEQ ID NO: 10 - DNA, 603 bp, Mytilus californianus
  source 1..603: mol_type = genomic DNA
gaccaaggatatggtggtggaagaagcattgggcatgtcggtggtggcagaggattgggatcaggcggtggtgccgtaagattggtattaggcggaggaggaggatccggcggtggtggagaaggaaaaggagatgggcgcgtgtggtgtacctgtcgtccaggacaatgtctaaaaggagaatttcgtttggacaaacaatgcagacttaccaaattgtttccttggaaatggtatacatgctgcaaggtgttaaaagtaactagccgtagttacgctggaggaatcagtggtggagctagatttggaggtggggcaggatatgggggcggagcaggatatttcggcggagctggatatggtggcagagctgtatatgtagttaaatctggatatggtggcggatctggttatggaggaggagctggatatggtaaaggatctggtcatggaggtggtgctggatatggaggaggatctggatatggcagtgggaattcatatattgttgtggcagatggaggttccggtggaggttctcgtggaggatattaatactaaatatgtttggtctgacaacacaaattatgtttaaaaatgaat